(19) United States
(12) Patent Application Publication
(10) Pub. No.: US 2014/0296729 A1
(43) Pub. Date: Oct. 2, 2014

(54) GAS MONITORING APPARATUSES, METHODS AND DEVICES

(71) Applicant: Shenzhen Mindray Bio-Medical Electronics Co., Ltd., Shenzhen (CN)

(72) Inventors: Zhonghua Liu, Shenzhen (CN); Liangjun Jiang, Shenzhen (CN); Youqiang Tu, Shenzhen (CN); Jian Cen, Shenzhen (CN); Fangyong Guan, Shenzhen (CN); Guangqi Huang, Shenzhen (CN); Ylva Sigun Israelsson, Shenzhen (CN)

(73) Assignee: Shenzhen Mindray Bio-Medical Electronics Co., Ltd.

(21) Appl. No.: 14/305,856

(22) Filed: Jun. 16, 2014

Related U.S. Application Data
(63) Continuation of application No. PCT/CN2012/086293, filed on Dec. 10, 2012.

(30) Foreign Application Priority Data
Dec. 15, 2011 (CN) 201110420113.3

(51) Int. Cl. A61B 5/08; A61B 5/00; A61B 5/087

(52) U.S. Cl. CPC A61B 5/082; A61B 5/087; A61B 5/7278; A61B 5/7203
USPC 600/532

(57) ABSTRACT

This disclosure provides gas monitoring apparatuses, methods and medical devices. A gas monitoring apparatus can include a gas concentration monitoring module, a respiratory mechanics monitoring module and a correction module. The correction module can determine a first correction value based on the gas ingredients and the gas concentration measured by the gas concentration monitoring module, so that the respiratory mechanics monitoring module can correct respiratory mechanics parameter(s) based on the first correction value. The correcting module can also determine a second correction value based on the pressure-related quantity measured by the respiratory mechanics monitoring module, so that the gas concentration monitoring module can correct gas concentration based on the second correction value.

### CROSS-REFERENCE

This application is a continuation of Patent Cooperation Treaty Application No. PCT/CN2012/086293, filed Dec. 10, 2012, which is hereby incorporated by reference.

### TECHNICAL FIELD

This disclosure relates to gas monitoring apparatuses, and particularly to apparatuses for performing gas monitoring on a patient's airway during anesthesia.

### BACKGROUND

There are two main types of gas monitoring, namely gas concentration monitoring and airway respiratory mechanics monitoring. Generally, gas concentration monitoring may be based on non-dispersive infrared (NDIR) analysis technique. That is, the gas concentration can be obtained by passing an infrared light of a particular waveband through a gas sample and then monitoring absorption of the infrared light by the gas sample. The selection of the infrared waveband and the number thereof may be related to the type of the gas monitored. Airway respiratory mechanics monitoring may be realized as follows: gas pressures can be first measured at several points within the airway to obtain three basic parameters including time, airway pressure and airway flow, and then some other parameters such as tidal volume, inspiration/expiration time rate (I:E rate) and positive end-expiratory pressure (PEEP) can be calculated to evaluate ventilation conditions within the airway.

Conventional approaches often perform gas concentration monitoring and airway respiratory mechanics monitoring separately, that is, gas concentration monitoring can be achieved via an independent monitoring module for analyzing gas ingredients and/or detecting gas concentration, while airway respiratory mechanics monitoring can be achieved via another independent monitoring module for obtaining three basic parameters including time, airway pressure and airway flow and then calculating some other parameters such as tidal volume, I:E rate and PEEP. The respective results from gas concentration monitoring and airway respiratory mechanics monitoring may then be separately outputted for consideration. During the monitoring process, changes in measurement environment may affect the monitoring results.

### SUMMARY OF THIS DISCLOSURE

This disclosure provides gas monitoring apparatuses, methods and devices that can improve accuracy for gas concentration monitoring and/or airway respiratory mechanics monitoring.

In one aspect, a gas monitoring apparatus can include a gas concentration monitoring module, a correction module and a respiratory mechanics monitoring module. The gas concentration monitoring module can extract gas from a patient's airway and measure and then output ingredients and concentration of the extracted gas. The correction module can be connected with an output port of the gas concentration monitoring module for obtaining the gas ingredients and the gas concentration outputted by the gas concentration monitoring module. The correction module can determine and then output a first correction value based on the gas ingredients and the gas concentration. The respiratory mechanics monitoring module can measure at least one respiratory mechanics parameter within the patient's airway. The respiratory mechanics monitoring module can be connected with an output port of the correction module for receiving the first correction value outputted by the correction module. Using a first pre-set correction scheme, the respiratory mechanics monitoring module can correct the respiratory mechanics parameter based on the first correction value and output the corrected respiratory mechanics parameter.

In some embodiments, the correction module can calculate a gas-flow property of mixed gas within the patient's airway based on the gas ingredients and the gas concentration, and determine the first correction value from a pre-determined measurement error curve based on the gas ingredients and the gas-flow property. The respiratory mechanics monitoring module may correct the respiratory mechanics parameter based on the first correction value and an airway flow.

In some embodiments, the correction module can also be connected with the respiratory mechanics monitoring module for obtaining a pressure-related quantity within the patient's airway measured by the respiratory mechanics monitoring module. The correction module can then determine a second correction value based on the pressure-related quantity and output the second correction value to the gas concentration monitoring module. Using a second pre-set correction scheme, the gas concentration monitoring module can correct the gas concentration based on the second correction value and output the corrected gas concentration.

In some embodiments, the pressure-related quantity may include an airway flow and a detection time. The correction module can determine a point suitable for calculation according to a waveform of the airway flow and send the detection time corresponding to the calculation point to the gas concentration monitoring module as the second correction value. The gas concentration corresponding to the detection time can be outputted by the gas concentration monitoring module.

In some embodiments, the correction module can determine stability of a gas flow according to a waveform of an airway flow, and evaluate whether to correct the gas concentration and/or to provide warning/prompt information based on the stability determined.

In another aspect, a gas monitoring apparatus can include a respiratory mechanics monitoring module, a correction module and a gas concentration monitoring module. The respiratory mechanics monitoring module can measure a pressure-related quantity within a patient's airway and calculate at least one respiratory mechanics parameter within the patient's airway. The correction module can be connected with the respiratory mechanics monitoring module for obtaining the pressure-related quantity within the patient's airway measured by the respiratory mechanics monitoring module. The correction module can then determine and output a second correction value based on the pressure-related quantity. The gas concentration monitoring module can extract gas from the patient's airway and measure and output a concentration of the extracted gas. Using a second pre-set correction scheme, the gas concentration monitoring module can correct the gas concentration based on the second correction value and output the corrected gas concentration.

In some embodiments, the pressure-related quantity can include an airway flow and a detection time. The correction module can determine a point suitable for calculation according to a waveform of the airway flow and output the detection time corresponding to the calculation point to the gas concentration monitoring module as the second correction value. The gas concentration corresponding to the detection time can be outputted by the gas concentration monitoring module.

In some embodiments, the correction module can determine stability of a gas flow according to the waveform of the airway flow. When the stability determined is lower than a first threshold, there may be no need to correct the gas concentration; when the stability determined is between the first threshold and a second threshold, the gas concentration can be corrected based on the second correction value using the second pre-set correction scheme; and when the stability determined is larger than the second threshold, warning/prompt information can be provided and the calculation of the gas concentration may be ended.

In yet another aspect, a gas monitoring method can include: extracting gas from a patient's airway and measuring ingredients and concentration of the extracted gas;

measuring a pressure-related quantity within the patient's airway and calculating at least one respiratory mechanics parameter within the patient's airway; determining a first correction value based on the gas ingredients and the gas concentration;

obtaining the pressure-related quantity measured within the patient's airway, determining a second correction value based on the pressure-related quantity and outputting the second correction value;

correcting the respiratory mechanics parameter based on the first correction value using a first pre-set correction scheme and outputting the corrected respiratory mechanics parameter; and

correcting the gas concentration based on the second correction value using a second pre-set correction scheme and outputting the corrected gas concentration.

In still another aspect, a medical device can include a gas monitoring apparatus described herein.

In this disclosure, the parameters obtained during gas concentration monitoring can be used for correcting the respiratory mechanics parameter(s) within the airway, so as to reduce the effects of gas type, gas density and viscosity and the like on the respiratory mechanics characteristic within the airway, thereby improving accuracy in respiratory mechanics measurements.

In this disclosure, the parameters obtained during respiratory mechanics monitoring can be used for correcting the gas concentration, so as to reduce the effects of varying airway conditions on the gas concentration, thereby improving accuracy in gas concentration measurements.

### BRIEF DESCRIPTION OF THE DRAWINGS

Following detailed descriptions of respective embodiments, this disclosure can be understood better when combining with these figures, in which the same structure is represented by the same reference sign. In the figures:

FIG. 1 is a block diagram for an embodiment of this disclosure;

FIG. 2 is a flow chart for correcting a respiratory mechanics parameter according to an embodiment of this disclosure;

FIG. 3 is a flow chart for correcting a gas concentration according to an embodiment of this disclosure.

### DETAILED DESCRIPTION

This disclosure is further described below in detail with reference to figures and specific implementations.

In clinical practice, gas concentration measurements within an airway are often affected by airway conditions. For example, when a test subject fails to have an open airway, expired and inspired gases may be mixed with each other in the airway, in which case the gas concentration measurements can be affected where measurement errors may occur. Further, measurements of respiratory mechanics parameters can be affected by gas type. Several different types of gas may exist in the airway, and gas density and viscosity may change as gas ingredients change. The change in gas density and viscosity can cause errors in the measurements of the respiratory mechanics parameters. In such situations, wrong measurement results may be obtained for respiratory mechanics characteristics, if not corrected.

In various embodiments, a gas concentration monitoring module and a respiratory mechanics monitoring module are used together to perform measurements on a patient's airway. Moreover, measurement results by the gas concentration monitoring module can be used for correcting measurement results by the respiratory mechanics monitoring module, and vice versa.

Referring now to FIG. 1, a gas monitoring apparatus can include a gas concentration monitoring module 10, a respiratory mechanics monitoring module 20 and a correction module 30, where the correction module 30 can be respectively connected with the gas concentration monitoring module 10 and the respiratory mechanics monitoring module 20. The correction module 30 can obtain gas ingredients and gas concentration from the gas concentration monitoring module 10 and then determine a correction value based on the gas ingredients and the gas concentration, so that the respiratory mechanics monitoring module 20 can correct its measurement result(s) (e.g., respiratory mechanics parameter 201) based on the correction value. The correction module 30 can also obtain at least one parameter representing airway conditions from the respiratory mechanics monitoring module 20 and then determine another correction value based on that parameter, so that the gas concentration monitoring module 10 can correct its measurement result(s) (e.g., gas concentration 101) based on said another correction value.

In an embodiment according to FIG. 1, the gas concentration monitoring module 10 can extract gas from a patient's airway and measure ingredients and/or concentration for the extracted gas. The respiratory mechanics monitoring module 20 can measure a pressure-related quantity within the patient's airway. For example, pressure sensors may be placed within the patient's airway to measure gas pressures at several points within the airway so as to obtain the pressure-related quantity. The pressure-related quantity can include at least one of the following three parameters: airway pressure, airway flow and time. In some cases, the pressure-related quantity can include all three parameters, i.e., airway pressure, airway flow and time. Respiratory mechanics parameters such as tidal volume, I:E rate and PEEP may be calculated based on those three parameters. The ventilation conditions within the patient's airway can then be evaluated via the respiratory mechanics parameters calculated. The correction module 30 can be respectively connected with the gas concentration monitoring module 10 and the respiratory mechanics monitoring module 20. On one hand the correction module 30 can obtain the gas ingredients and the gas concentration outputted from the gas concentration monitoring module 10, and on the other hand the correction module 30 can also obtain the pressure-related quantity within the patient's airway measured by the respiratory mechanics monitoring module 20. The correction module 30 can determine a first correction value based on the gas ingredients and the gas concentration and output the first correction value to the gas concentration monitoring module 10. The correcting module 30 can also determine a second correction value based on the pressure-related quantity and output the second correction value to the respiratory mechanics monitoring module 20. The respiratory mechanics monitoring module 20 may receive the first correction value from the correction module 30, correct the respiratory mechanics parameter(s) based on the first correction value using a first pre-set correction scheme, and output the corrected respiratory mechanics parameter(s). The first correction scheme can be formed based on fluid mechanics, for example a compensation based on the Reynolds number. Other suitable correction schemes can also be formed based on fluid mechanics that are capable of correcting respiratory mechanics parameter(s) with gas ingredients and gas concentration measured. The gas concentration monitoring module 10 can receive the second correction value from the correction module 30, correct the gas concentration based on the second correction value using a second pre-set correction scheme, and output the corrected gas concentration.

In another embodiment, the gas monitoring apparatus only corrects the respiratory mechanics measurement(s) using the parameters obtained by gas concentration monitoring. Alternatively, the gas monitoring apparatus only corrects the gas concentration measurement using the parameters obtained by respiratory mechanics monitoring.

The gas concentration monitoring module 10 and the respiratory mechanics monitoring module 20 can be integrated into one device, or they can be used separately in two devices. The gas concentration monitoring module 10 and the respiratory mechanics monitoring module 20 can have independent power supplies and/or processors respectively, or they may share a common power supply and/or a common processor.

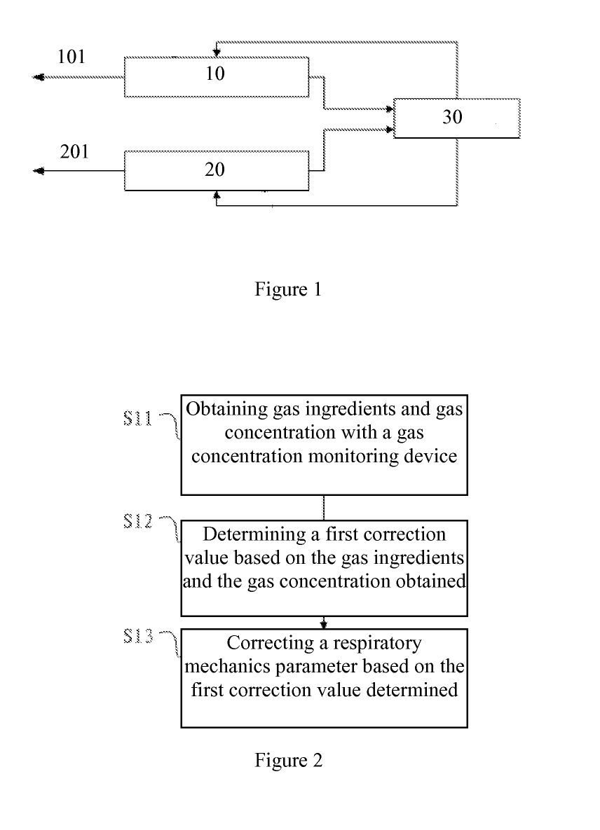

An example method for monitoring gas concentration within a patient's airway using the gas monitoring apparatus described herein is shown in FIG. 2. The method can include the following steps.

In step S11, a gas concentration monitoring module can extract gas from a patient's airway and perform measurement on the gas to calculate gas ingredients and/or gas concentration. The gas concentration monitoring module may extract the gas from the airway by certain flow, evaluate absorption changes of the extracted gas, identify the gas type based on the changes, and calculate the gas concentration. Any suitable existing or future apparatus for measuring gas ingredients and/or gas concentration can be employed as the gas concentration monitoring module.

In step S12, a correction module can determine a first correction value based on the gas ingredients and the gas concentration measured. Specifically, the correction module can calculate a gas-flow property of mixed gas within the patient's airway based on the gas ingredients and the gas concentration, where the gas-flow property can at least include density and viscosity of the mixed gas. The first correction value can then be determined from a pre-stored measurement error curve based on the gas ingredients and the gas-flow property.

In step S13, a respiratory mechanics monitoring module can correct a respiratory mechanics parameter based on the first correction value using a first pre-set correction scheme and output the corrected respiratory mechanics parameter.

In an embodiment, the correction module can obtain the gas type and the gas concentration within the airway from the measurements of the gas concentration monitoring module, determine the physical parameters such as the density and the viscosity of the mixed gas within the airway, and then correct the respiratory mechanics measurement, along with data such as current flow obtained by the respiratory mechanics monitoring module. In some cases, the respective measurement error curves f suitable for various gas monitoring conditions can be established through many experiments performed with an inert gas. The measurement error curve for a particular gas type can be corrected by a coefficient coff, based on the measurement error curve of the inert gas. The correction module can determine a correction coefficient err_coff (f*coff) (i.e., the first correction value) from the measurement error curve f, based on the obtained parameters such as the gas type and the gas density. This correction coefficient err_coff (f*coff) can then be used to correct the respiratory mechanics measurement, where the first correction scheme used can be, for example, that the corrected respiratory mechanics parameter is obtained by multiplying a preliminary respiratory mechanics parameter by the correction coefficient.

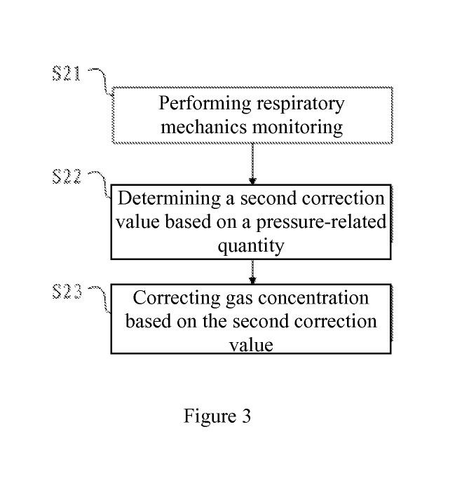

Based on the above-described gas monitoring apparatuses, an example method for monitoring respiratory mechanics within a patient's airway using the gas monitoring apparatus described herein is shown in FIG. 3. The method can include the following steps.

In step S21, a respiratory mechanics monitoring module may measure a pressure-related quantity within the patient's airway and calculate at least one respiratory mechanics parameter therein. Any suitable existing or future apparatus for measuring airway conditions can be employed as the respiratory mechanics monitoring module. The respiratory mechanics monitoring module may obtain some parameters including real-time pressures and pressure differences based on pressure changes within the airway and calculate real-time gas flow. Other parameters may be calculated based on time, pressure and flow.

In step S22, the correction module can obtain the pressure-related quantity measured within the patient's airway, determine a second correction value based on the pressure-related quantity and output the second correction value. The correction module may determine a point suitable for calculation according to a waveform of an airway flow and send the detection time corresponding to the calculation point to the gas concentration monitoring module as the second correction value. In an embodiment, the correction module may evaluate gas conditions within the airway based on the measurement results of the respiratory mechanics monitoring module. For example, the correction module may evaluate stability of the gas flow based on the waveform of the airway flow, evaluate whether there is turbulent flow and/or gas mixing within the airway, and based on such evaluation correct/process the respiratory mechanics parameters calculated by the respiratory mechanics monitoring module. In normal respiratory situations, the flow curve may have typical inspiration and expiration waveforms. If the inspiration and expiration waveforms disappear or become disordered, turbulent flow may have occurred. In this case, warning and/or prompt information can be provided, depending on the extent of the turbulent flow; alternatively, a point suitable for calculation can be determined based on the waveform feature and the detection time corresponding to the calculation point may be provided to the gas concentration monitoring apparatus. In an embodiment, the correction module may evaluate flow conditions of the gas flow (e.g., stability of the gas flow) within the airway based on the waveform shape of an airway flow. For example, the Reynolds number can be detected within the airway, and the stability of the gas flow and whether there is turbulent flow can be evaluated based on the Reynolds number. In fluid mechanics, the Reynolds number is a ratio between fluid inertia force and fluid viscous force. When the Reynolds number is relatively small, the viscous force may have greater effects on the flow field than the inertia force, in which case the disturbance on the flow rate may be damped by the viscous force, and the flow can be relatively stable as laminar flow. When the Reynolds number is relatively large, the inertia force may have greater effects on the flow field than the viscous force, in which case the flow can be relatively unstable, and small changes in the flow rate can increase, thereby creating disordered turbulent flow. Typically, the Reynolds number can be calculated based on fluid property (density and viscosity), fluid velocity and characteristic length/characteristic dimension. For fluid flow in a tube, the Reynolds number can be defined as follows:

Re=(ρVD)/μ=(VD)/v=(QD)/vA

where V is an average flow rate (m/s), D is the diameter of the tube (e.g., characteristic length) (m), μ is dynamic viscosity of the fluid (Pa□s or N□s/m□), v is kinematic viscosity (v=μ/ρ) (m□/s), ρ is fluid density (kg/m³), Q is volume flow (m³/s) and A is a cross-sectional area (m²).

When the Reynolds number is smaller than a first threshold, there may be no need for gas concentration correction; when the Reynolds number is between the first threshold and a second threshold, the gas concentration may be corrected based on the second correction value using the second pre-set correction scheme; and when the Reynolds number is larger than the second threshold, warning and/or prompt information may be provided where the calculation of the gas concentration would be stopped and no gas concentration value would be outputted. In an embodiment, the first threshold can be set as about 2100, while the second threshold can be set as about 4000. When the Reynolds number is smaller than about 2100, laminar flow (also called viscous flow or linear flow) can exist, where there may be no need to correct the gas concentration. If the Reynolds number is between about 2100 and about 4000, transition flow may exist, and the gas concentration obtained by the gas concentration monitoring module can be corrected using the respiratory mechanics parameter(s) obtained from the respiratory mechanics monitoring module. When the Reynolds number is greater than about 4000, turbulent flow (also called disorder flow or disturbed flow) may exist, at which point warning and/or prompt information may be provided and the gas concentration monitoring module may be controlled to stop outputting the gas concentration value.

In step S23, the gas concentration monitoring module can correct the gas concentration based on the second correction value using the second pre-set correction scheme, and then output the corrected gas concentration. As an example, the second correction scheme can be as follows: among its calculation results, the gas concentration monitoring module may determine a gas concentration value corresponding to the detection time outputted by the correction module, and then output such gas concentration value as the final result.

Steps S11 and S12 can be carried out on the airway of a single patient during the same time period. In some embodiments, the gas concentration monitoring module and the respiratory mechanics monitoring module may be used simultaneously for monitoring the airway. However, those skilled in the art should understand that the gas concentration monitoring module and the respiratory mechanics monitoring module can have certain time differences during data sampling as long as the potential error would be within an allowable range.

The gas monitoring apparatuses described herein can be applied to medical devices. When the medical devices are equipped with displays, the corrected results from the gas concentration monitoring module and the respiratory mechanics monitoring module may be displayed.

The medical devices can be a patient bedside machine such as a patient monitor, an anesthesia machine and/or a respiratory machine

This disclosure is described above as detailed illustrations with reference to specific implementations, while this disclosure should not be limited to these illustrations. For those of ordinary skills in the art, various conclusions or equivalents may be made without departing from the concept of this disclosure, while such conclusions or equivalents should be deemed to be included within the scope of this disclosure.

1. A gas monitoring apparatus, comprising:
a gas concentration monitoring module for extracting gas from a patient's airway, and measuring and outputting gas ingredients and gas concentration;
a correction module that is connected with an output port of the gas concentration monitoring module for obtaining the gas ingredients and the gas concentration outputted by the gas concentration monitoring module; wherein the correction module is capable of determining a first correction value based on the gas ingredients and the gas concentration, and outputting the first correction value; and
a respiratory mechanics monitoring module for measuring at least one respiratory mechanics parameter within the patient's airway; wherein the respiratory mechanics monitoring module is connected with an output port of the correction module for receiving the first correction value outputted by the correction module; and wherein the respiratory mechanics monitoring module is capable of correcting the respiratory mechanics parameter based on the first correction value using a first pre-set correction scheme and outputting the corrected respiratory mechanics parameter.

2. The gas monitoring apparatus of claim 1, wherein the correction module is capable of calculating a gas-flow property of mixed gas within the patient's airway based on the gas ingredients and the gas concentration, and determining the first correction value from a pre-determined measurement error curve based on the gas ingredients and the gas-flow property; and wherein the respiratory mechanics monitoring module corrects the respiratory mechanics parameter based on the first correction value and an airway flow.

3. The gas monitoring apparatus of claim 1, wherein the correction module is also connected with the respiratory mechanics monitoring module for obtaining a pressure-related quantity within the patient's airway measured by the respiratory mechanics monitoring module; and the correction module is capable of determining a second correction value based on the pressure-related quantity and outputting the second correction value to the gas concentration monitoring module; and wherein the gas concentration monitoring module is capable of correcting the gas concentration based on the second correction value using a second pre-set correction scheme and outputting the corrected gas concentration.

4. The gas monitoring apparatus of claim 3, wherein the pressure-related quantity comprises an airway flow and a detection time; wherein the correction module is capable of determining a point suitable for calculation according to a waveform of the airway flow and outputting a detection time corresponding to the calculation point to the gas concentration monitoring module as the second correction value; and wherein the gas concentration monitoring module outputs the gas concentration corresponding to the detection time.

5. The gas monitoring apparatus of claim 3, wherein the correction module is capable of determining stability of a gas flow according to a waveform of an airway flow, and evaluating whether to correct the gas concentration and/or to provide warning/prompt information based on the stability determined.

6. A gas monitoring apparatus, comprising:
a respiratory mechanics monitoring module for measuring a pressure-related quantity within a patient's airway and calculating at least one respiratory mechanics parameter within the patient's airway;
a correction module that is connected with the respiratory mechanics monitoring module for obtaining the pressure-related quantity measured by the respiratory mechanics monitoring module; wherein the correction module is capable of determining a second correction value based on the pressure-related quantity, and outputting the second correction value; and
a gas concentration monitoring module for extracting gas from the patient's airway, and measuring and outputting gas concentration; wherein the gas concentration monitoring module is capable of correcting the gas concentration based on the second correction value using a second pre-set correction scheme and outputting the corrected gas concentration.

7. The gas monitoring apparatus of claim 6, wherein the pressure-related quantity comprises an airway flow and a detection time; wherein the correction module is capable of determining a point suitable for calculation according to a waveform of the airway flow and outputting a detection time corresponding to the calculation point to the gas concentration monitoring module as the second correction value; and wherein the gas concentration monitoring module outputs the gas concentration corresponding to the detection time.

8. The gas monitoring apparatus of claim 7, wherein the correction module is capable of determining stability of a gas flow according to the waveform of the airway flow; and wherein when the stability determined is lower than a first threshold, the gas concentration is not corrected; when the stability determined is between the first threshold and a second threshold, the gas concentration is corrected based on the second correction value using the second pre-set correction scheme; and, when the stability determined is larger than the second threshold, warning/prompt information is provided and the calculation of the gas concentration is ended.

9. A gas monitoring method, comprising:
extracting gas from a patient's airway and measuring the gas to calculate gas ingredients and gas concentration;
measuring a pressure-related quantity within the patient's airway and calculating at least one respiratory mechanics parameter within the patient's airway;
determining a first correction value based on the gas ingredients and the gas concentration;
determining a second correction value based on the pressure-related quantity and outputting the second correction value;
correcting the respiratory mechanics parameter based on the first correction value using a first pre-set correction scheme and outputting the corrected respiratory mechanics parameter; and
correcting the gas concentration based on the second correction value using a second pre-set correction scheme and outputting the corrected gas concentration.

10. A medical device comprising a gas monitoring apparatus according to claim 1.

11. The medical device of claim 10, wherein the correction module is capable of calculating a gas-flow property of mixed gas within the patient's airway based on the gas ingredients and the gas concentration, and determining the first correction value from a pre-determined measurement error curve based on the gas ingredients and the gas-flow property; and wherein the respiratory mechanics monitoring module corrects the respiratory mechanics parameter based on the first correction value and an airway flow.

12. The medical device of claim 10, wherein the correction module is also connected with the respiratory mechanics monitoring module for obtaining a pressure-related quantity within the patient's airway measured by the respiratory mechanics monitoring module; and the correction module is capable of determining a second correction value based on the pressure-related quantity and outputting the second correction value to the gas concentration monitoring module; and wherein the gas concentration monitoring module is capable of correcting the gas concentration based on the second correction value using a second pre-set correction scheme and outputting the corrected gas concentration.

13. The medical device of claim 12, wherein the pressure-related quantity comprises an airway flow and a detection time; wherein the correction module is capable of determining a point suitable for calculation according to a waveform of the airway flow and outputting a detection time corresponding to the calculation point to the gas concentration monitoring module as the second correction value; and wherein the gas concentration monitoring module outputs the gas concentration corresponding to the detection time.

14. The medical device of claim 12, wherein the correction module is capable of determining stability of a gas flow according to a waveform of an airway flow, and evaluating whether to correct the gas concentration and/or to provide warning/prompt information based on the stability determined.

15. A medical device comprising a gas monitoring apparatus according to claim 6.

16. The medical device of claim 15, wherein the pressure-related quantity comprises an airway flow and a detection time; wherein the correction module is capable of determining a point suitable for calculation according to a waveform of the airway flow and outputting a detection time corresponding to the calculation point to the gas concentration monitoring module as the second correction value; and wherein the gas concentration monitoring module outputs the gas concentration corresponding to the detection time.

17. The medical device of claim 16, wherein the correction module is capable of determining stability of a gas flow according to the waveform of the airway flow; and wherein when the stability determined is lower than a first threshold, the gas concentration is not corrected; when the stability determined is between the first threshold and a second threshold, the gas concentration is corrected based on the second correction value using the second pre-set correction scheme; and, when the stability determined is larger than the second threshold, warning/prompt information is provided and the calculation of the gas concentration is ended.
